(19) United States
(12) Patent Application Publication
(10) Pub. No.: US 2016/0030753 A1
(43) Pub. Date: Feb. 4, 2016

(54) MULTI-ELECTRODE NEURAL PROTHESIS SYSTEM

(71) Applicant: LAWRENCE LIVERMORE NATIONAL SECURITY, LLC, Livermore, CA (US)

(72) Inventors: Kedar G. Shah, San Francisco, CA (US); Phillipe Tabada, Rseville, CA (US); Terri L. Delima, Livermore, CA (US); Satinderpall S. Pannu, Pleasanton, CA (US)

(21) Appl. No.: 14/777,332

(22) PCT Filed: Mar. 17, 2014
(86) PCT No.: PCT/US14/30768

§ 371 Date: Sep. 15, 2015

Related U.S. Application Data
(60) Provisional application No. 61/802,477, filed on Mar. 16, 2013.

(51) Int. Cl. A61N 1/375

(52) U.S. Cl. CPC A61N 1/3754

(57) ABSTRACT

A hermetic electronics package of a multi-electrode neural prosthesis system includes a metal case, a feedthrough construction having an electrically insulating substrate and an array of electrically conductive feedthroughs extending through it, with the electrically insulating substrate connected to the open end of the metal case to form a hermetically sealed enclosure. And a set of electronic components is located within the hermetically sealed enclosure and operably connected to the feedthroughs of the feedthrough construction so as to electrically communicate outside the package. And a demultiplexer is fsoperatively connected to demultiplex a single signal into multiple signals prior to being transmitted through the feedthroughs.

### CROSS-REFERENCE TO RELATED APPLICATIONS

This patent document claims the benefits and priorities of U.S. Provisional Application No. 61/802,477, filed on Mar. 16, 2013, hereby incorporated by reference.

### FEDERALLY SPONSORED RESEARCH OR DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### TECHNICAL FIELD

This patent document relates to hermetically sealed electronic packages and devices, and in particular to a multi-electrode neural prosthesis system having a hermetically sealed electronics package with a de-multiplexer to transmit/receive multiple electrical impulses through a set of electrical feedthroughs connecting to an external electrode array, for high density electrode array operation.

### BACKGROUND

Electrically-active implantable bio-medical devices (such as for example pacemakers, cochlear implants, and neural prosthetics) are increasing in popularity due to the potential of continuous monitoring, instantaneous and directed delivery of treatments, reduction of treatment costs, and unique treatment options. However, because many of the component materials used in such devices are not bio-compatible, that is, they are toxic to the body and can induce undesirable biological reactions, it is critical to hermetically seal the non-bio-compatible components (e.g. CMOS, passive components, batteries) in a bio-compatible material, so that the body does not have a cyto-toxic response. Hermetic sealing also helps protects electrical components from damage due to moisture and the corrosive environment in the body.

FIG. 1 shows a schematic illustration of a general approach to hermetically encapsulating implantable devices, such as 10, where non-bio-compatible components and materials 11, such as electronics, are encapsulated in a hermetically sealed package 12 made of bio-compatible materials. In this arrangement, an array of hermetic electrically conducting feedthroughs 13 is provided on an electrically insulating portion 14 of the package 12 for use as electrical conduits which allow communication of electrical signals between the body and electronics within the package.

And U.S. Pat. No. 7,881,799 describes a retinal prosthetic device having a hermetically sealed electronic package that contains a single side of the package that consists of electrical feedthroughs to transfer electrical signals between the device electronics and the polymer electrode array that attaches to the retina.

One limitation of current devices is the limited number of electrodes and thus the restriction in the number of electrical signals that can be transmitted through the electronics package, whether signals are transmitted out of the package or received into the package. State-of-the-art bio-compatible ceramics with electrical feedthroughs are limited in density by the inability to create closely spaced, small diameter vias that can be filled with metal paste. In applications such as retinal prosthetics, for example, it is critical to increase the number of electrodes, which has a direct impact on the resolution of the image that the patient can see.

Similarly, some limitations of current neural implants are: 1. low number of electrical channels for stimulation of neural tissue, 2. large electronics package size to accommodate electronics, 3. lack of high-density interconnect technologies; and 4. limitations of wireless data and power telemetry to increase number of channels.

In order to improve the performance of implantable devices, it is advantageous to provide multiple electrical signals through a fixed number of electrically conductive feedthroughs so as to increase feedthrough density and interactivity with the implanted medium.

### SUMMARY

The technology described in this patent document includes hermetic electronics packages, devices, and systems with high-density hermetic electrical feedthroughs and methods for fabricating the same.

In one example implementation, the present invention includes a hermetic electronics package comprising: a metal case with an open end; a feedthrough construction having an electrically insulating substrate and an array of electrically conductive feedthroughs extending therethrough, said electrically insulating substrate connected to the open end of the metal case so as to form a hermetically sealed enclosure; a set of electronic components located within the hermetically sealed enclosure and operably connected to the feedthroughs of the feedthrough construction on as to electrically communicate outside the package, and a demultiplexer operatively connected to demultiplex a single signal into multiple signals prior to being transmitted through the feedthroughs.

In another example implementation, the present invention includes a multi-electrode neural prosthesis system comprising: a metal case with an open end; a feedthrough construction having an electrically insulating substrate and an array of electrically conductive feedthroughs extending therethrough, said electrically insulating substrate connected to the open end of the metal case so as to form a hermetically sealed enclosure; a set of electronic components located within the hermetically sealed enclosure and operably connected to the feedthroughs of the feedthrough construction so as to electrically communicate outside the package, and a de-multiplexer operatively connected to demultiplex a single signal into multiple signals prior to being transmitted through thefs feedthroughs, and located in the hermetically sealed enclosure as one of said electronic components and directly connected to the feedthroughs, wherein the electronics components includes a driver connected by interconnects to an interconnect board for further connection by interconnects to both passive components and the de-multiplexer.

And in another example implementation, the present invention includes a multi-electrode neural prosthesis system comprising: a metal case with an open end; a feedthrough construction having an electrically insulating substrate and an array of electrically conductive feedthroughs extending therethrough, said electrically insulating substrate connected to the open end of the metal case so as to forma hermetically sealed enclosure; a set of electronic components located within the hermetically sealed enclosure and operably connected to the feedthroughs of the feedthrough construction so as to electrically communicate outside the package, and a de-multiplexer operatively connected to demultiplex a single signal into multiple signals prior to being transmitted through the feedthroughs, and located in the hermetically sealed enclosure as one of said electronic components and directly connected to the feedthroughs, wherein the electronics components includes a driver, wherein the de-multiplexer is located outside the hermetically sealed enclosure and embedded into a microelectrode array connected to the feedthroughs.

These and other implementations and various features and operations are described in greater detail in the drawings, the description and the claims.

The present invention is generally directed to the design and method of manufacturing a multi-electrode neural prosthesis system that is fully wireless and long-term implantable in the human body. In the present invention, a hermetically-sealed package contains active circuitry (a combination of passive components, electronic chips, interconnects, and antennas for power and data telemetry). The components are assembled in a manner that allows multiple electrical impulses to be transmitted through a set of hermetic electrical feedthroughs to the outside of the package. A polymer-based multi-electrode array is electrically attached to the electronic package such that it interfaces with, and stimulates or records from living tissue and cells.

Such a device and system may be used for, but is not limited to, retinal prostheses, neural prostheses, neural stimulators, and a variety of implantable bio-medical devices (e.g. coclear implant) that stimulate or record from live tissue, such as wireless implantable systems, implantable bio-medical devices, such as for deep brain stimulation, or neural disorder treatment. In this manner, the present invention addresses the problem described in the Background of enabling high density feedthrough operation and scalability by demultiplexing a signal into multiple signals transmitted through electrical feedthroughs. The resulting device would exhibit the same bio-compatibility and hermeticity specifications, however it has the ability to substantially increase the number of electrical signals that can be simultaneously transmitted from the device. This enables a retinal device, for example, to equivalently increase the resolution visible to patients, which significantly improves the existing technology.

For bio-medical implant applications in particular, substrate materials that have high bio-compatibility and are capable of being hermetically sealed to implantable metal packages are preferred. Example bio-compatible electrically conductive substrate materials that may be used include: titanium and its alloys, such as surgical grade titanium—Ti6Al4V, Ti6Al4V ELI (‘extra low interstitials’) and niobium and alloys. While bio-compatible electrically conductive metal substrates are preferred in bio-medical implant applications, if the electrically conductive substrate material was further coated with an insulating material then any electrical conductor may be used, such as but not limited to platinum and alloys (such as platinum-iridium); iridium and alloys; ruthenium and alloys; Nitinol (Ti—Ni); palladium and alloys; rhodium and alloys, gold and alloys; copper and alloys, aluminum and alloys, surgical grade stainless steel such as 316LVM; p- or n-type doped silicon; etc. Electrical resistance of individual wires may be less than about 500 ohms. And it is also notable that various types of electrically insulating materials may be used as well, e.g. glass, polymer, or ceramic insulators. For example, the electrically insulating material may be a bio-compatible electrically insulating material, such as for example sealing glasses such as Pyrex, non-leaded glass, boro-silicate glass, glass-frit powder or paste, glasses or ceramics containing one or more of B₂O₃, CaO, BaO, SiO₂, La₂ O₃, Al_{2O3}, Li₂O₃, Ti_{O2}.

### BRIEF DESCRIPTION OF THE DRAWINGS

FIG. 1 is a schematic view of an implantable device illustrating a common approach to encapsulating non-bio-compatible component materials in a bio-compatible sealed package,

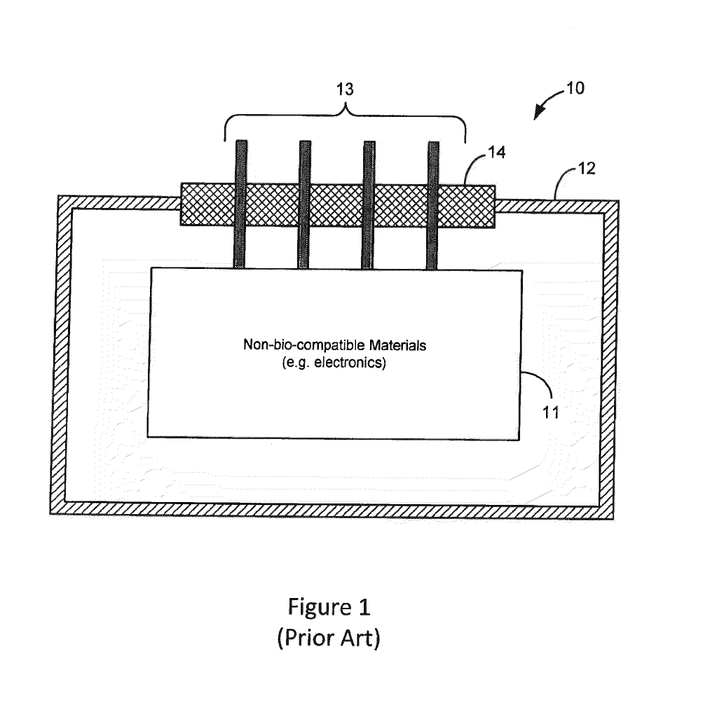

FIG. 2 is a schematic view of a first example embodiment of the hermetic electronic package and system of the present invention.

FIG. 3 is a schematic view of a second example embodiment of the hermetic electronic package and systfsem of the present invention.

FIG. 4 is a schematic view of a third example embodiment of the hermetic electronic package and system of the present invention.

### DETAILED DESCRIPTION

The present invention is generally directed to a multi-electrode neural prosthesis system having a hermetic electronic package with an electrical feedthrough configuration that may be used for electrically active, implantable bio-medical devices. In the present invention, the channel count is significantly increased (such as, by a factor of 2, 4, 8, or 16) by incorporating a de-multiplexing chip, which takes a single electrical input signal and converts it into multiple outputs. The input signal operates at a higher frequency than the outputs, and hence, de-multiplexing the signal does not degrade signal quality or affect the performance of the neural prosthetic.

The hermetically-sealed package contains a set of electronic components (e.g. a combination of passive components, electronic chips, interconnects, antennas for power and data telemetry, cables, etc.). And a plurality of electrically conductive feedthroughs are provided on a wall of the package to enable electronic components housed inside to electrically communicate outside the package. In particular, a single or multiple polymer-based multi-electrode arrays (which for example may contain electrodes that interface with living tissue and cells) may be attached to the feedthroughs of the electronic package.

Generally, four example embodiments of the present invention are herein described, wherein all of the embodiments share a common set of components: a driver chip which converts the incoming power and data signals into individual electrical signals; passive components, such as resistors, capacitors, and diodes; a de-multiplexer chip as explained above; hermetic feedthroughs: an array of electrical feedthroughs that permit the electrical signals to be transported outside the electronics package; and interconnects—various forms of interconnects to electrically connect the above components, in the form of wire-bonds, metalized traces, interconnect boards with feedthroughs and metallization, and conductive epoxies applied to connect various components.

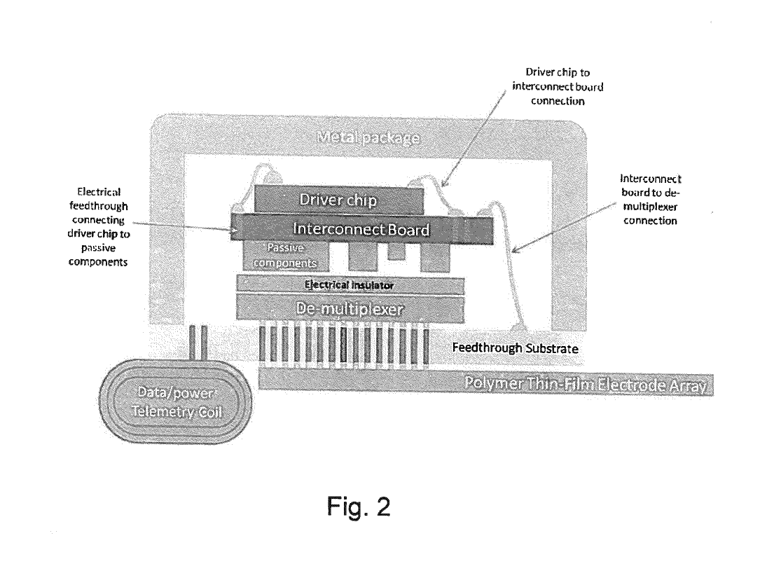

FIG. 2 shows a first example embodiment of the multi-electrode neural prosthesis system of the present invention, where the driver chip, de-multiplexer and the passive components are all assembled inside the electronics package. An interconnect board (electrically insulated substrate with electrical feedthroughs and lithographically defined metal pattern on both sides) is used to connect the passive components to the driver chip, and to connect the driver chip to the de-multiplexer. An electrically insulating shim may be used to separate the passive components from the de-multiplexer. All of the electronic components are hermetically sealed in a metal package (e.g. metal case), and the electrical signals exit this package through the feedthrough substrate that contains an array of hermetic electrical feedthroughs, The polymer thin-film electrode array (also known as the microelectrode array), and the antenna are electrically connected to the external side of the electronics package.

In FIG. 2, the metal case is shown having one end (lower end) that is capped with a electrical feedthrough construction. The electrical feedthrough constructions have an electrically insulating substrate, and a plurality of electrically conductive feedthroughs extending through it. The electrically insulating substrate may be made of for example, a ceramic with multiple metal-filled vias for the feedthroughs. And the electrically insulating substrate in particular are brazed (e.g. melting a braze alloy), bonded, or otherwise hermetically joined to the metal casing, so as to form the hermetically sealed enclosure which houses a set of electronic components on the inside of the device, including for example, integrated circuit chips (electronic drivers, de-multiplexers, etc), passive electrical components (resistors, capacitors, diodes, etc), interconnects (wire-bonds, electrical traces), cables, and antenna (for wireless data and power telemetry).

And a data/power telemetry coil is also shown on the exterior of the package and connected to feedthroughs. It is appreciated that electronic component assembly may involve various techniques known in the art, such as for example, thermo-compression flipchip bonding of the IC chips, conductive epoxies to attach passive components, wire-bonding, and lithographically patterned conductive traces.

Furthermore, on the outside of the package, a single or multiple polymer electrode array may be provided and connected to the feedthroughs from opposite sides of the package. In particular, FIG. 2 shows a polymer thin film electrode array connected to the feedthroughs of the top feedthrough construction. The polymer electrode array consists of a multitude of conductive traces sandwiched between multiple polymer layers. In particular, the electrode array may have a plurality of traces extending between electrodes at a lead end and a connector end. The lead end of the polymer electrode array terminates in the electrodes that interface with the implanted medium, e.g. tissue (for electrical recording or stimulation).

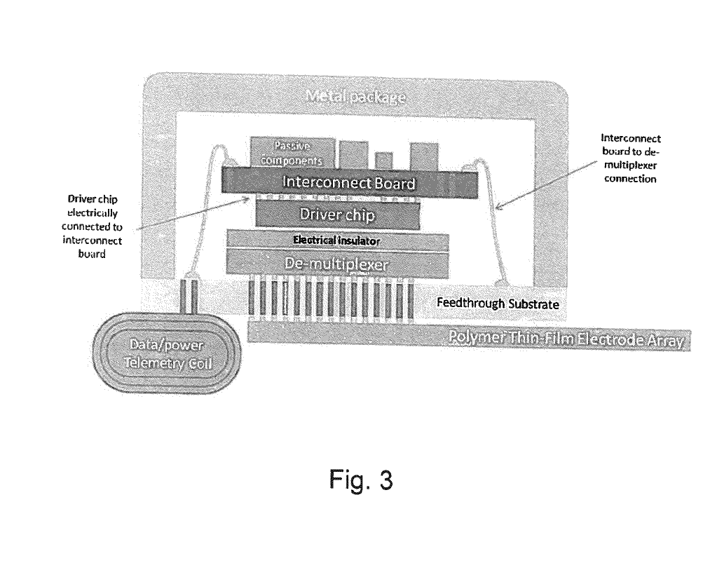

FIG. 3 shows a second example embodiment having similar components as the first example embodiment in FIG. 2. However, the orientation of the driver chip and the passive components are switched. The metal pads on the driver chip face the interconnect board, and is electrically connected to the interconnect board such that all the outputs from the driver chip can be connected as inputs to the de-multiplexer. The de-multiplexer outputs its electrical signals directly to the microelectrode array, which is attached outside the package.

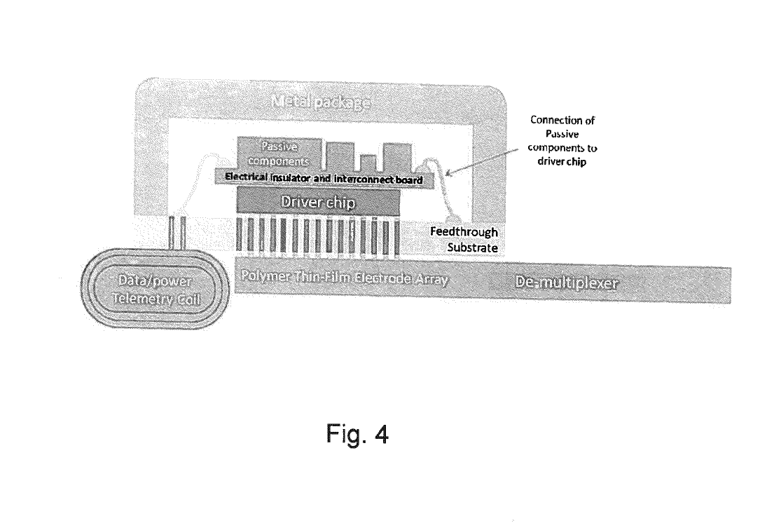

FIG. 4 shows a third example embodiment where the de-multiplexer is coated with a hermetic bio-compatible coating and electrically embedded into the microelectrode array (outside the electronics package). This enables a fewer number of channels to be routed from the electronics package to the electrode array. By integrating the de-multiplexer closer to the electrode array region, the polymer cable dimensions can be minimized. The electronics package configuration is simplified to include the driver chip, passive components mounted on an interconnect board, and the necessary interconnects to electrically connect all the components to each other.

In a fourth example embodiment of the system of the present invention, the passive comments are integrated into the driver or de-multiplexer chip to reduce the space requirements of the electronics package.

In all of the embodiments described above, a combination of microfabrication processes may be used for assembling the entire device. For example: hermetic feedthrough substrates may be manufactured by filling vias in a ceramic substrate with gold or platinum conductors. The top and bottom surface of the ceramic are metalized and patterned using lithographic processes. The substrate may be attached to the metal package using brazing. The metal package may consist of a ring and a lid, in which case they are attached using laser welding. The thin-film electrode array may consists of metal layers and traces sandwiched between layers of polymer (such as silicone, polyimide and parylene). The driver chip and the de-multiplexer may be fabricated using standard CMOS manufacturing methods. Passive components may be obtained as commercial off the shelf (COTS) items, and may be attached to the interconnect board or other substrates with conductive epoxies or solder. The driver chip or the de-multiplexer may be electrically connected to the other components using flip-chip bonding of conductive stud bumps, by conductive epoxy bumps, or by wire-bonding between metal pads on each substrate. The microelectrode array may flip-chip bonded to the can using conductive epoxy bumps printed on both the ceramic feedthrough substrate and the microelectrode array. And epoxies may be used after many of the above processes to provide mechanical stability, or electrical isolation.

It is notable that hermetically sealed packages with electrical feedthroughs is commonly used by many companies in the bio-medical device industry to separate non-bio-compatible components from bodily tissue. However, electrical feedthroughs are also heavily used in the semiconductor industry to interconnect electronic chips. And electrical feedthroughs may also be used in other applications, such as separating sensors or electronics from harsh environments in the field. It is appreciated therefore that while bio-compatible materials are preferred for use as one or both of the electrically conductive substrate/feedthroughs and electrically insulating materials of the present invention when used in bio-medical implant applications, other non-bio-compatible materials may be used in the alternative for other non-bio-medical applications. The challenge in all these applications, however, remains the same, that is to create very high-density hermetic feedthroughs using materials that are compatible with the environment of application.

Although the description above contains many details and specifics, these should not be construed as limiting the scope of the invention or of what may be claimed, but as merely providing illustrations of some of the presently preferred embodiments of this invention. Other implementations, enhancements and variations can be made based on what is described and illustrated in this patent document. The features of the embodiments described herein may be combined in all possible combinations of methods, apparatus, modules, systems, and computer program products. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural and functional equivalents to the elements of the above-described preferred embodiment that are known to those of ordinary skill in the art are expressly incorporated herein by reference and are intended to be encompassed by the present claims. Moreover, it is not necessary for a device to address each and every problem sought to be solved by the present invention, for it to be encompassed by the present claims. Furthermore, no element or component in the present disclosure is intended to be dedicated to the public regardless of whether the element or component is explicitly recited in the claims. No claim element herein is to be construed under the provisions of 35 U.S.C. 112, sixth paragraph, unless the element is expressly recited using the phrase “means for.”

We claim:

1. A hermetic electronics package comprising:
a metal case with an open end;
a feedthrough construction having an electrically insulating substrate and an array of electrically conductive feedthroughs extending therethrough, said electrically insulating substrate connected to the open end of the metal case so as to form a hermetically sealed enclosure;
a set of electronic components located within the hermetically sealed enclosure and operably connected to the feedthroughs of the feedthrough construction so as to electrically communicate outside the package, and
a demultiplexer operatively connected to demultiplex a single signal into multiple signals prior to being transmitted through the feedthroughs.

2. The hermetic electronics package of claim 1,
wherein the de-multiplexer is located in the hermetically sealed enclosure as one of said electronic components and directly connected to the feedthroughs.

3. The hermetic electronics package of claim 2,
wherein the electronics components includes a driver connected by interconnects to an interconnect board for further connection by interconnects to both passive components and the de-multiplexer.

4. The hermetic electronics package of claim
wherein the electronics components includes a driver directly connected to an interconnect board which is itself connected by an interconnect to the de-multiplexer.

5. The hermetic electronics package of claim 1,
wherein the de-multiplexer is located outside the hermetically sealed enclosure and embedded into a microelectrode array connected to the feedthroughs.

6. The hermetic electronics package of claim 1,
wherein the electronics components includes passive components integrated into one of a driver chip and the de-multiplexer.

7. The hermetic electronics package of claim 1,
wherein the electrically conductive feedthroughs are a bio-compatible metal.

8. The hermetic electronics package of claim 7,
wherein the electrically conductive bio-compatible metal is selected from the group consisting of titanium, platinum, iridium, ruthenium, niobium, palladium, gold, stainless steel, p- or n-type doped silicon, and alloys thereof.

9. The hermetic electronics package of claim 1,
wherein the electrically insulating material is selected from the group consisting of glass, polymer, ceramic, and other dielectric materials.

10. The hermetic electronics package of claim 1,
wherein the electrically insulating substrate is a bio-compatible material.

11. The hermetic electronics package of claim 1,
wherein the electrically insulating bio-compatible material is selected from the group consisting of sealing glasses, non-leaded glass, boro-silicate glass, glass-frit powder or paste, and glasses or ceramics containing one or more of B₂O₃, CaO, BaO, SiO₂, La₂O₃, Al₂O₃, Li₂O₃, TiO₂.

12. A multi-electrode neural prosthesis system comprising:
a metal case with an open end;
a feedthrough construction having an electrically insulating substrate and an array of electrically conductive feedthroughs extending therethrough, said electrically insulating substrate connected to the open end of the metal case so as to form a hermetically sealed enclosure;
a set of electronic components located within the hermetically sealed enclosure and operably connected to the feedthroughs of the feedthrough construction so as to electrically communicate outside the package, and
a de-multiplexer operatively connected to demultiplex a single signal into multiple signals prior to being transmitted through thefs feedthroughs, and located in the hermetically sealed enclosure as one of said electronic components and directly connected to the feedthroughs, wherein the electronics components includes a driver connected by interconnects to an interconnect board for further connection by interconnects to both passive components and the de-multiplexer.

13. A multi-electrode neural prosthesis system comprising:
a metal case with an open end;
a feedthrough construction having an electrically insulating substrate and an array of electrically conductive feedthroughs extending therethrough, said electrically insulating substrate connected to the open end of the metal case so as to faun a hermetically sealed enclosure;
a set of electronic components located within the hermetically sealed enclosure and operably connected to the feedthroughs of the feedthrough construction so as to electrically communicate outside the package, and
a de-multiplexer operatively connected to demultiplex a single signal into multiple signals prior to being transmitted through the feedthroughs, and located in the hermetically sealed enclosure as one of said electronic components and directly connected to the feedthroughs, wherein the electronics components includes a driver, wherein the de-multiplexer is located outside the hermetically sealed enclosure and embedded into a microelectrode array connected to the feedthroughs.
